(19) United States
(12) Patent Application Publication
(10) Pub. No.: US 2026/0354999 A1
(43) Pub. Date: Nov. 12, 2026

(54) CROSSING ELEMENT FOR ORTHOPEDIC AND SPORTS AID DEVICES

(71) Applicant: BAUERFEIND AG, Zeulenroda-Triebes (DE)

(72) Inventors: Gerald STIER, Zeulenroda-Triebes (DE); Sandro HEBENSTREIT, Zeulenroda-Triebes (DE); Beatrix BAUERFEIND-JOHNSON, Zeulenroda-Triebes (DE)

(21) Appl. No.: 19/138,025

(22) PCT Filed: Nov. 20, 2023
(86) PCT No.: PCT/EP2023/082436

§ 371 Date: Jun. 11, 2025

(30) Foreign Application Priority Data
Dec. 13, 2022 (DE) 10 2022 213 572.5

(51) Int. Cl. A61F 5/01; A61F 5/02

(52) U.S. Cl. CPC A61F 5/0127; A61F 5/0123; A61F 5/013; A61F 5/028

(57) ABSTRACT

The present invention relates to the technical field of orthopedic and sports aid devices. In particular, the invention provides a means for the controlled and, in particular, low-friction crossing of tensioning elements in orthopedic and sports aid devices applied to the human or animal body.

The present invention relates to the technical field of orthopedic and sports aid devices. In particular, the invention provides a means for the controlled and, in particular, low-friction crossing of at least one tensioning element in orthopedic and sports aid devices applied to the human or animal body.

A particular problem in the guidance of tensioning elements in orthopedic and sports aid devices consists at the points where different tensioning elements or sections of a tensioning element cross each other. Without control of the course of the at least one tensioning element at the crossing points, the different tensioning elements or the sections of the connected tensioning element come into contact with each other, resulting in frictional forces which significantly impair the transmission of force and cause undesirable wear of the tensioning elements. In the case of an unguided crossing of different tensioning elements or of sections of a tensioning element, forces acting on the crossing point from outside may cause the at least one tensioning element to become blocked. In connection with this, the mobility of orthopedic and sports aid devices may also be impaired. In the prior art, this is prevented by guiding tensioning elements in such a way as to avoid crossing points, in particular by using deflection buckles, rings, or straps. The aim of the present invention is therefore to provide means for the controlled and, in particular, low-friction crossing of at least one tensioning element in orthopedic and sports aid devices.

The present invention solves the underlying technical problem by means of the subject matter of the independent claims.

The present invention relates in particular to a crossing element for orthopedic or sports aid devices, comprising at least one first guide section for receiving a tensioning element of the aid device and at least one second guide section for receiving a tensioning element of the aid device, wherein the at least two guide sections of the crossing element are designed and arranged relative to one another such that a tensioning element guided in the first guide section and a tensioning element guided in the second guide section cross each other without contact at at least one point. In particular, the at least one tensioning element guided by the guide sections of the crossing element runs in different planes at least in the at least one crossing point.

In a particularly preferred embodiment of the present invention, the crossing element for orthopedic and sports aid devices is a crossing element for orthoses, in particular for a back orthosis, hip orthosis, knee joint orthosis, elbow orthosis, arm orthosis, or ankle joint orthosis.

According to a preferred embodiment of the present invention, the crossing element for orthopedic and sports aid devices is a crossing element for bandages, in particular for a back bandage, hip bandage, knee joint bandage, elbow bandage, arm bandage, or ankle joint bandage.

In a further preferred embodiment of the present invention, the crossing element for orthopedic and sports aid devices is a crossing element for sports aid devices, in particular for gloves and shoes, preferably for sports shoes, in particular for jogging shoes, hiking shoes, cycling shoes, ski shoes, or snowboard boots.

In a preferred embodiment, the crossing element is made of at least one plastic or one metal, particularly a light metal. The at least one plastic is preferably selected from the group consisting of polystyrene (PS), polycarbonate (PC), polypropylene (PP), polyethylene terephthalate (PET), cycloolefins (COC), polyamide (PA), polyketone (PK), acrylonitrile-butadiene-styrene (ABS), polyethylene (PE), thermoplastic polyurethane (TPU), thermoplastic elastomers (TPE), and mixtures thereof.

In a preferred embodiment, the crossing element is made of at least two, preferably at least three, preferably at least four, different materials, in particular of at least two, preferably at least three, preferably at least four, different plastics.

According to the invention, the crossing element comprises at least two guide sections. In a preferred embodiment of the present invention, it may be provided that the crossing element comprises at least three, preferably at least four, preferably at least five, guide sections. Preferably, the crossing element according to the invention comprises at most five, preferably at most four, preferably at most three, guide sections. In an especially preferred embodiment, the crossing element comprises two, preferably three, preferably four, preferably five, guide sections.

Accordingly, in the crossing element according to the invention, at least two tensioning elements, in particular two tensioning elements, of an orthopedic or sports aid device cross each other without contact. According to the invention, it may be provided that at least three, at least four or at least five tensioning elements, in particular three, four or five tensioning elements, of an orthopedic or sports aid device cross each other without contact in the crossing element.

According to a preferred embodiment of the present invention, at least one guide section of the crossing element, particularly the first and/or second guide section of the crossing element, is a guide channel. The guide channel may be open or closed.

It is preferred that at least one guide section, in particular the first and/or the second guide section, in particular the first and/or the second guide channel, of the crossing element is designed as an open guide channel, in particular as a groove. It is especially preferred that at least one guide section, in particular the first and/or the second guide section, in particular the first and/or the second guide channel, of the crossing element is designed as a groove tapering towards the at least one crossing point.

In a preferred embodiment of the present invention, at least one guide section, in particular the first and/or second guide section, is shaped such that a tensioning element guided in the at least one guide section, in particular in the first and/or second guide section, is at least partially encompassed. In a further embodiment, at least one guide section, in particular the first and/or second guide section, can be shaped such that a tensioning element guided in the at least one guide section, in particular in the first and/or second guide section, is completely encompassed. According to the invention, it may be provided that a tensioning element guided in at least one guide section, in particular in the first and/or second guide section, of the crossing element is encompassed over the entire length of the at least one guide section, in particular of the first and/or second guide section, or in sections, in particular partially or completely encompassed.

According to the invention, it may also be provided that at least one guide section, in particular the first and/or the second guide section, in particular the first and/or the second guide channel, of the crossing element is a closed channel.

It is especially preferred that the first guide section is designed such that a tensioning element of the aid device guided in the first guide section runs in the at least one crossing point above a tensioning element of the aid device guided in the second guide section.

In a further preferred embodiment, the first guide section is designed such that a tensioning element of the aid device guided in the first guide section runs in the at least one crossing point below a tensioning element of the aid device guided in the second guide section.

In a particularly preferred embodiment of the present invention, the guide sections of the crossing element, in particular the first and second guide sections, are located in different planes.

The guide sections of the crossing element, particularly the first and second guide sections of the crossing element, are preferably located in different planes at least in the region of at least one crossing point of a tensioning element of the aid device guided in the first guide section with a tensioning element of the aid device guided in the second guide section.

The guide sections of the crossing element, in particular the first and second guide sections of the crossing element, are preferably located in all crossing points of the tensioning elements of the aid device, in particular in all crossing points of a tensioning element of the aid device guided in the first guide section with a tensioning element of the aid device guided in the second guide section, in different planes, in particular in at least two different planes.

In a preferred embodiment of the present invention, the guide sections of the crossing element are arranged in different planes. It is especially preferred that the guide sections of the aid device are arranged in different planes, particularly in the region of at least one crossing point, preferably in the region of at least two crossing points, preferably in the region of at least three crossing points, particularly in the region of all crossing points, of the tensioning elements of the aid device. The guide sections of the crossing element are preferably arranged in at least two, preferably at least three, preferably at least four, preferably at least five, different planes.

According to a preferred embodiment of the present invention, the first guide section is arranged above the second guide section of the crossing element. It is preferred that the first guide section is arranged above the second guide section at least in the region of at least one crossing point of a tensioning element of the aid device guided in the first guide section with a tensioning element of the aid device guided in the second guide section. According to this preferred embodiment of the invention, at the at least one crossing point, a tensioning element guided in the first guide section of the crossing element is guided without contact above a tensioning element guided in the second guide section of the crossing element.

It is especially preferred that the first guide section is located at least in the region of at least one crossing point of a tensioning element of the aid device guided in the first guide section with a tensioning element of the aid device guided in the second guide section at least 0.25 cm, preferably at least 0.5 cm, preferably at least 0.75 cm, preferably at least 1 cm, preferably at least 1.25 cm, preferably at least 1.5 cm, preferably at least 1.75 cm, preferably at least 2 cm, preferably at least 2.25 cm, preferably at least 2.5 cm, above the second guide section.

In a further preferred embodiment of the invention, the first guide section is located at least in the region of at least one crossing point of a tensioning element of the aid device guided in the first guide section with a tensioning element of the aid device guided in the second guide section at most 3 cm, preferably at most 2.75 cm, preferably at most 2.5 cm, preferably at most 2 cm, preferably at most 1.75 cm, preferably at most 1.5 cm, preferably at most 1.25 cm, preferably at most 1 cm, preferably at most 0.75 cm, preferably at most 0.5 cm, above the second guide section.

According to a preferred embodiment, the first guide section is located at least in the region of at least one crossing point of a tensioning element of the aid device guided in the first guide section with a tensioning element of the aid device guided in the second guide section 0.25 cm to 3 cm, preferably 0.25 cm to 2.5 cm, preferably 0.5 cm to 2 cm, preferably 0.5 cm to 1.5 cm, preferably 0.75 cm to 1 cm, above the second guide section.

Preferably, at least one guide section, in particular the first or second guide section, comprises at least one ramp, preferably two ramps. In a preferred embodiment of the present invention, the tensioning element of the aid device running in the first guide section is guided by means of at least one ramp, preferably by means of two ramps, above the tensioning element of the aid device running in the second guide section.

In an especially preferred embodiment of the present invention, the first guide section comprises two ramps, wherein the second guide section runs between the two ramps of the first guide section so that a tensioning element running in the first guide section is guided by means of the two ramps above a tensioning element running in the second guide section.

In a further embodiment of the present invention, at least one guide section, in particular the first or second guide section, comprises at least one bridge. It is preferred that the tensioning element of the aid device running in the first guide section is guided by means of the bridge above the tensioning element of the aid device running in the second guide section.

In a preferred embodiment of the present invention, the guide sections of the crossing element, in particular the first and second guide sections, run orthogonally to each other. It is especially preferred that the tensioning element of the aid device guided in the first guide section and the tensioning element of the aid device guided in the second guide section cross orthogonally at at least one point. According to the invention, the tensioning elements of the aid device guided in the guide sections of the crossing element, in particular the tensioning element of the aid device guided in the first guide section and the tensioning element of the aid device guided in the second guide section, cross each other without contact.

It is especially preferred that the first and second guide sections of the crossing element are arranged relative to each other such that a tensioning element of the aid device guided in the first guide section and a tensioning element of the aid device guided in the second guide section cross in the crossing element with an angle of intersection a of at least 20°, preferably at least 25°, preferably at least 30°, preferably at least 35°, preferably at least 40°, preferably at least 45°, preferably at least 50°, preferably at least 55°, preferably at least 60°, preferably at least 65°, preferably at least 70°, preferably at least 75°, preferably at least 80°, preferably at least 85°, preferably 90°.

According to a preferred embodiment of the present invention, the first and second guide sections of the crossing element are arranged relative to each other such that a tensioning element of the aid device guided in the first guide section and a tensioning element of the aid device guided in the second guide section cross in the crossing element with an angle of intersection a of at most 90°, preferably at most 85°, preferably at most 80°, preferably at most 75°, preferably at most 70°, preferably at most 65°, preferably at most 60°, preferably at most 55°, preferably at most 45°, preferably at most 40°, preferably at most 35°, preferably at most 30°, preferably at most 25°.

In a further preferred embodiment of the present invention, the first and second guide sections of the crossing element are arranged relative to each other such that a tensioning element of the aid device guided in the first guide section and a tensioning element of the aid device guided in the second guide section cross in the crossing element with an angle of intersection α of 20° to 90°, preferably 25° to 90°, preferably 30° to 90°, preferably 35° to 90°, preferably 40° to 90°, preferably 45° to 90°, preferably 50° to 90°, preferably 55° to 90°, preferably 60° to 90°, preferably 65° to 90°, preferably 70° to 90°, preferably 75° to 90°, preferably 80° to 90°, preferably 85° to 90°.

In a further preferred embodiment of the present invention, the first and second guide sections of the crossing element are arranged relative to each other such that a tensioning element of the aid device guided in the first guide section and a tensioning element of the aid device guided in the second guide section cross in the crossing element with an angle of intersection a of 20° to 90°, preferably 25° to 85°, preferably 30° to 80°, preferably 35° to 75°, preferably 40° to 70°, preferably 45° to 65°, preferably 50° to 60°.

In a preferred embodiment of the present invention, the at least two guide sections of the crossing element are designed and arranged relative to one another such that the tensioning elements of the aid device running in the guide sections cross each other without contact at at least two, preferably at least three, preferably at least four, points.

According to a further preferred embodiment of the present invention, the at least two guide sections of the crossing element are designed and arranged relative to one another such that the tensioning elements of the aid device guided in the guide sections cross each other without contact at at most four, preferably at at most three, preferably at at most two, points.

It is especially preferred that the at least two guide sections of the crossing element are designed and arranged relative to one another such that the tensioning elements of the aid device guided in the guide sections cross without contact at one point, preferably at two points, preferably at three points, preferably at four points.

According to a preferred embodiment of the present invention, the crossing element comprises at least one cover. Especially preferred, the crossing element comprises at least one cover at least above the at least one crossing point of a tensioning element guided in the first guide section with a tensioning element guided in the second guide section.

The cover in the region of the crossing of the tensioning elements guided in the guide sections of the crossing element, particularly in the region of the crossing of the tensioning element guided in a first guide section with the tensioning element guided in a second guide section, advantageously protects the at least one tensioning element of the aid device crossing in the crossing element from external influences. In particular, the cover provides protection against undesirable pressure on the at least one crossing point of the at least one tensioning element of the aid device.

In a preferred embodiment of the present invention, the cover of the crossing element also comprises at least one further guide section in which a tensioning element of the aid device can be guided, in particular can be guided without contact above the further tensioning elements running in the crossing element. According to this embodiment, it may be provided that tensioning elements which are guided in the at least one first guide section and the at least one second guide section of the crossing element and in the at least one further guide section of the cover cross in at least three different planes, in particular cross without contact.

According to a further preferred embodiment of the present invention, the cover of the crossing element is removable. According to the invention, it may also be provided that the cover is not removable, in particular that the cover cannot be removed from the crossing element without being destroyed.

It is preferred that the cover of the crossing element is rotatably mounted. The rotatability of the cover allows forces acting on the at least one tensioning element of the aid device to be distributed more evenly.

According to the present invention, the tensioning elements guided in the at least two guide sections of the crossing element, in particular the tensioning element guided in the first guide section and the tensioning element guided in the second guide section, may be the same tensioning element of the aid device, in particular a single connected tensioning element of the aid device. According to the invention, it may also be provided that the tensioning elements guided in the at least two guide sections of the crossing element, in particular the tensioning element guided in the first guide section and the tensioning element guided in the second guide section, are different tensioning elements of the aid device, in particular tensioning elements of the aid device which are not connected to one another.

The tensioning elements guided in the at least two guide sections of the crossing element, particularly the tensioning element guided in the first guide section of the crossing element and the tensioning element guided in the second guide section of the crossing element, are preferably the same tensioning element of the aid device. According to this embodiment, the tensioning elements guided in the at least two guide sections of the crossing element, in particular the tensioning element guided in the first guide section of the crossing element and the tensioning element guided in the second guide section of the crossing element, are a single connected tensioning element of the aid device, which crosses at least once without contact in the crossing element according to the invention. Correspondingly, it may be provided, for example, that a single tensioning element runs in an aid device in such a way that different sections of the tensioning element cross without contact in the crossing element according to the invention.

In a further embodiment of the present invention, the tensioning elements guided in the at least two guide sections of the crossing element, in particular the tensioning element guided in the first guide section of the crossing element and the tensioning element guided in the second guide section of the crossing element, are different tensioning elements of the aid device. According to this embodiment, the tensioning elements guided in the at least two guide sections of the crossing element, in particular the tensioning element guided in the first guide section of the crossing element and the tensioning element guided in the second guide section of the crossing element, are tensioning elements of the aid device which are not connected to one another. According to the invention, it can therefore be provided that the tensioning element guided in the first guide section and the tensioning element guided in the second guide section are two tensioning elements of an aid device which are not connected to one another and which cross one another without contact in the crossing element according to the invention.

According to a preferred embodiment, the at least one tensioning element, in particular the tensioning element guided in the first guide section and/or the tensioning element guided in the second guide section, is elastic, partially elastic or inelastic.

In a preferred embodiment of the present invention, the at least one tensioning element, in particular the tensioning element of the aid device guided in the first guide section of the crossing element and/or the tensioning element of the aid device guided in the second guide section of the crossing element, is selected from the group consisting of tensioning or pulling strap, tensioning or pulling rope, tensioning or pulling band, and limb-like tensioning or pulling element.

Preferably, the at least one tensioning element, particularly the tensioning element of the aid device guided in the first guide section and/or the tensioning element of the aid device guided in the second guide section, is a tensioning or pulling strap. In a further embodiment, the at least one tensioning element, particularly the tensioning element of the aid device guided in the first guide section and/or the tensioning element of the aid device guided in the second guide section, is a tensioning or pulling rope. Preferably, the at least one tensioning element, particularly the tensioning element of the aid device guided in the first guide section and/or the tensioning element of the aid device guided in the second guide section, is a tensioning or pulling strap. In a further embodiment of the present invention, the at least one tensioning element, particularly the tensioning element of the aid device guided in the first guide section and/or the tensioning element of the aid device guided in the second guide section, is a limb-like tensioning or pulling element.

In a preferred embodiment of the present invention, it may be provided that the at least one tensioning element, in particular the tensioning element guided in the first guide section and/or the tensioning element guided in the second guide section, of the aid device is completely or partially, in particular in sections, encased.

In an especially preferred embodiment of the present invention, the at least one tensioning element, particularly the tensioning element guided in the first guide section and/or the tensioning element guided in the second guide section, is a Bowden cable.

In a preferred embodiment of the present invention, the crossing element is integrated into an orthopedic or sports aid device. It is especially preferred that the crossing element cannot be separated from the orthopedic or sports aid device without being destroyed.

According to a further preferred embodiment of the present invention, the crossing element can be removed from the orthopedic or sports aid device.

The present invention also relates to an orthopedic or sports aid device comprising at least one crossing element according to the invention.

According to a preferred embodiment, the orthopedic or sports aid device comprises at least two, preferably at least three, preferably at least four, crossing elements. Preferably, the orthopedic or sports aid device comprises at most ten, preferably at most nine, preferably at most eight, preferably at most seven, preferably at most six, preferably at most five, preferably at most four, preferably at most three, preferably at most two, crossing elements. In a further preferred embodiment, the orthopedic or sports aid device comprises two, preferably three, preferably four, preferably five, crossing elements.

In a preferred embodiment of the present invention, the orthopedic or sports aid device is selected from the group consisting of back orthosis, back bandage, hip orthosis, hip bandage, knee joint orthosis, knee joint bandage, elbow orthosis, elbow bandage, arm orthosis, arm bandage, ankle joint orthosis, ankle joint bandage, gloves, and shoes. Especially preferred is the orthopedic or sports aid device selected from the group consisting of back orthosis, back bandage, hip orthosis, hip bandage, knee joint orthosis, knee joint bandage, elbow orthosis, elbow bandage, arm orthosis, arm bandage, ankle joint orthosis and ankle joint bandage.

The orthopedic or sports aid device is preferably a back orthosis. In a preferred embodiment of the invention, the orthopedic or sports aid device is a back bandage. According to a particularly preferred embodiment, the orthopedic or sports aid device is a hip orthosis. Preferably, the orthopedic or sports aid device is a hip bandage. According to the invention, it may also be provided that the orthopedic or sports aid device is a knee joint orthosis. The orthopedic or sports aid is preferably a knee joint bandage. According to a preferred embodiment, the orthopedic or sports aid is an arm orthosis. The orthopedic or sports aid is preferably an arm bandage. In a further embodiment, the orthopedic or sports aid is an ankle joint orthosis. According to a further preferred embodiment of the invention, the orthopedic or sports aid is an ankle joint bandage. The orthopedic or sports aid is preferably a glove. In a particularly preferred embodiment of the present invention, the orthopedic or sports aid device is a shoe, in particular a sports shoe, for example a jogging shoe, a hiking shoe, a cycling shoe, a ski shoe, or snowboard boots.

According to a preferred embodiment of the present invention, the at least one crossing element of the orthopedic or sports aid device is rotatably mounted. It is especially preferred that the at least one crossing element of the orthopedic or sports aid device is rotatably mounted about a rotation axis, in particular about a central rotation axis. Due to the rotatability of the crossing element and thus of the first and second guide sections, shear and/or pulling forces acting on the aid device can be better absorbed and distributed by the tensioning elements. For example, when the orthopedic or sports aid device is in place, increasing tension on the tensioning elements may cause a slight change in the shape of the orthopedic or sports aid device, which may also change the position of the tensioning elements crossing each other without contact in the crossing element. The rotatability of the crossing element can compensate for changes in the shape of the aid device and distribute the forces acting on the aid device by the tensioning elements evenly.

The present invention also relates to the use of a crossing element according to the invention for crossing at least one tensioning element of an orthopedic or sports aid device without contact.

The embodiments described in connection with the crossing element according to the invention and the statements made also relate mutatis mutandis to the orthopedic or sports aid device according to the invention and the use of the crossing element according to the invention.

Further embodiments of the present invention are apparent from the dependent claims.

In the context of the present invention, the term “crossing element” refers to a structure at or within which a crossing of at least two tensioning elements can be achieved without contact. According to the invention, this may be a structure comprising one, two or more parts.

In the context of the present invention, an “orthopedic or sports aid device” is understood to be an orthopedic aid device that can be applied to the human or animal body for the treatment and correction of congenital malpositions, for the prevention and therapy of chronic diseases, and for the treatment of injuries to the musculoskeletal system. According to the invention, the designation also covers an aid device that can be applied to the human or animal body for supporting, stabilizing, and/or protecting the musculoskeletal system, particularly for supporting, stabilizing, and/or protecting the ligaments, joints, and/or muscles during increased physical activity, particularly during sports. According to the invention, the term “sports aid device” also covers sports articles that can be worn on the body, such as gloves and shoes. The “orthopedic or sports aid device” is preferably a support device that can be worn externally on the human or animal body and at least partially encompasses at least one extremity. According to the present invention, the “orthopedic or sports aid device” is especially preferred in the form of an orthosis or a bandage.

In the context of the present invention, the term “guide section” refers to a spatially definable part of a crossing element that is suitable for guiding a tensioning element, i.e., for limiting the mobility of the tensioning element within certain limits. In a particularly preferred embodiment, the “guide section” of the crossing element according to the invention is designed in the form of an open or closed guide channel, particularly in the form of a groove, preferably in the form of a groove tapering toward the crossing point.

In the context of the present invention, a “tensioning element” is an elastic, partially elastic, or inelastic structure that is suitable for absorbing, transmitting, and/or distributing forces, particularly tensile and pulling forces. The “tensioning element” is especially preferred to be a tensioning or pulling strap, a tensioning or pulling rope, a tensioning or pulling band, or a limb-like tensioning or pulling element. According to the invention, the “tensioning element” guided in the first guide section of the aid device and the “tensioning element” guided in the second guide section may be a single connected “tensioning element” of an aid device or different, unconnected “tensioning elements” of the aid device.

In the context of the present invention, the terms “comprising” and “having” are understood to mean that, in addition to the elements explicitly covered by these terms, further elements not explicitly mentioned may be added. In the context of the present invention, these terms are also understood to mean that only the explicitly mentioned elements are included and that no further elements are present. In this particular embodiment, the meaning of the terms “comprising” and “having” is synonymous with the term “consisting of”. Furthermore, the terms “comprising” and “having” also encompass compositions which, in addition to the explicitly mentioned elements, comprise further elements which are not mentioned but which are of a functionally and qualitatively subordinate nature. In this embodiment, the terms “comprising” and “having” are synonymous with the term “consisting essentially of”.

In the context of the present invention, the term “and/or” means that all members of a group connected by the term “and/or” are disclosed both alternatively to each other and cumulatively in any combination. For the expression “A, B, and/or C,” this means that the following disclosure content is to be understood: a) A or B or C or b) (A and B) or c) (A and C) or d) (B and C) or e) (A and B and C).

The present invention is illustrated below using exemplary figures. The invention is not limited to these embodiments.

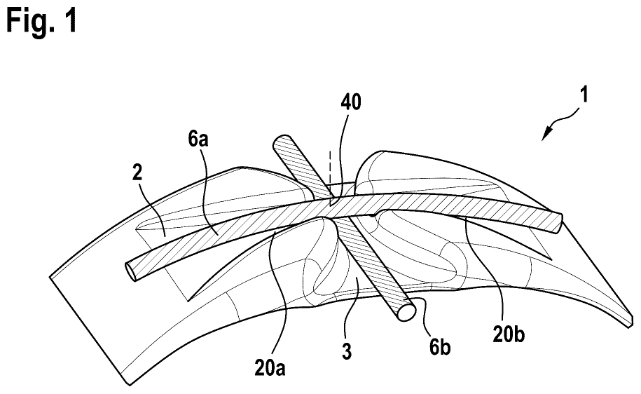

FIG. 1 shows a perspective view of a crossing element (1) according to the invention with two guide sections (2, 3) for receiving and guiding at least one tensioning element (6a, 6b) which crosses without contact at the crossing point (40).

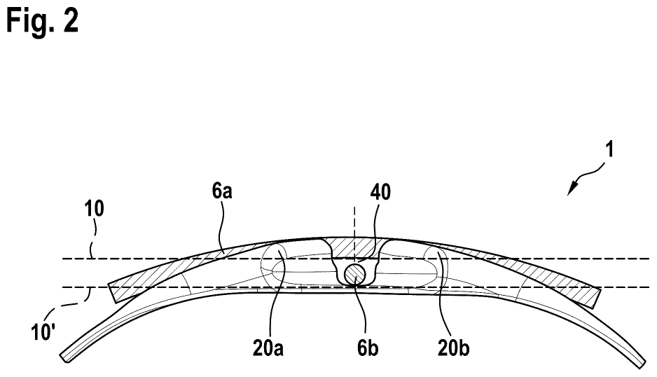

FIG. 2 shows a side view of a crossing element (1) according to the invention. This figure shows that the tensioning element (6a) located in the first guide section (2) is guided in the crossing point (40) by means of two ramps (20a, 20b) in a plane (10) without contact above the tensioning element (6b) located in the second guide section (3) and in a plane (10′) below it.

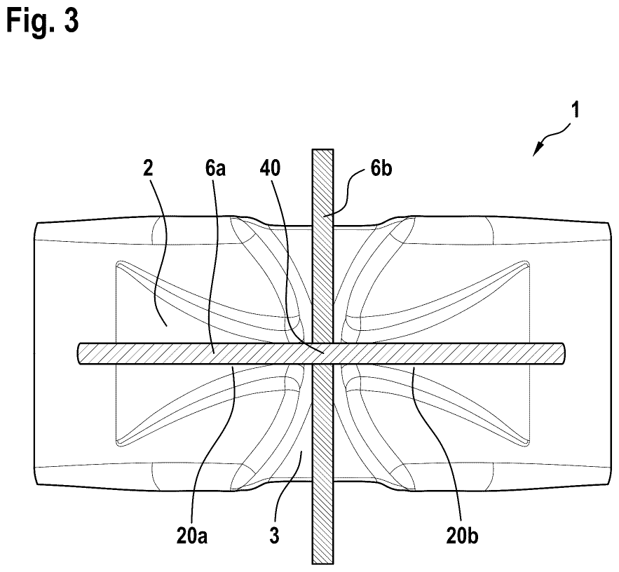

FIG. 3 shows a top view of a crossing element (1) according to the invention. In the embodiment of the crossing element (1) shown, the two guide sections (2, 3) are each designed as a groove tapering toward the crossing point (40). Such a design of the guide sections (2, 3) allows the tensioning element (6a) located in the first guide section (2) and the tensioning element (6b) located in the second guide section (3) a certain freedom of movement in the distal regions of the crossing element (1) and increasingly restricts the freedom of movement towards the crossing point (4) in order to prevent the at least one tensioning element (6a, 6b) from slipping and to ensure that the elements cross without contact.

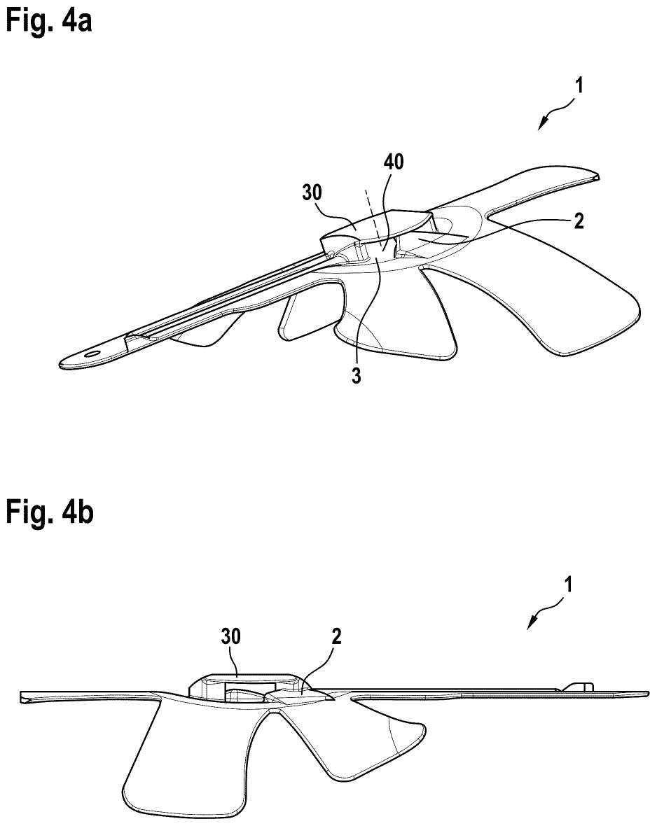

FIG. 4a shows a perspective view of a crossing element (1) according to the invention for a back orthosis or back bandage with two guide sections (2, 3) and a cover (30) extending above the crossing point (40). In the embodiment shown, the two guide sections (2, 3) are each designed to be wide enough that tensioning or pulling straps, for example, can cross at the crossing point (40) without contact. The cover (30) serves to protect the tensioning elements from external influences, in particular to prevent pressure from acting on the tensioning elements at the crossing point (40). FIG. 4b shows a side view of the crossing element (1) for a back orthosis or back bandage illustrated in FIG. 4a.

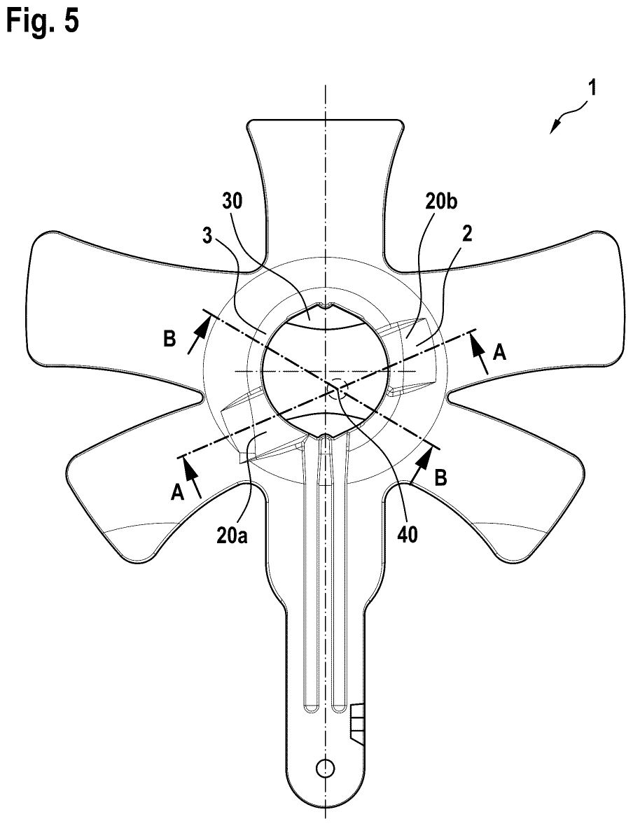

FIG. 5 shows a top view of the embodiment of the crossing element (1) according to the invention shown in FIGS. 4a and 4b. The two wide guide sections (2, 3) allow tensioning or pulling belts to cross at a crossing point (40) which is protected by a cover (30). A tensioning element guided in the first guide section (2) crosses a tensioning element guided in the second guide section (3) without contact by means of the ramps (20a, 20b) flanking the crossing point (40).

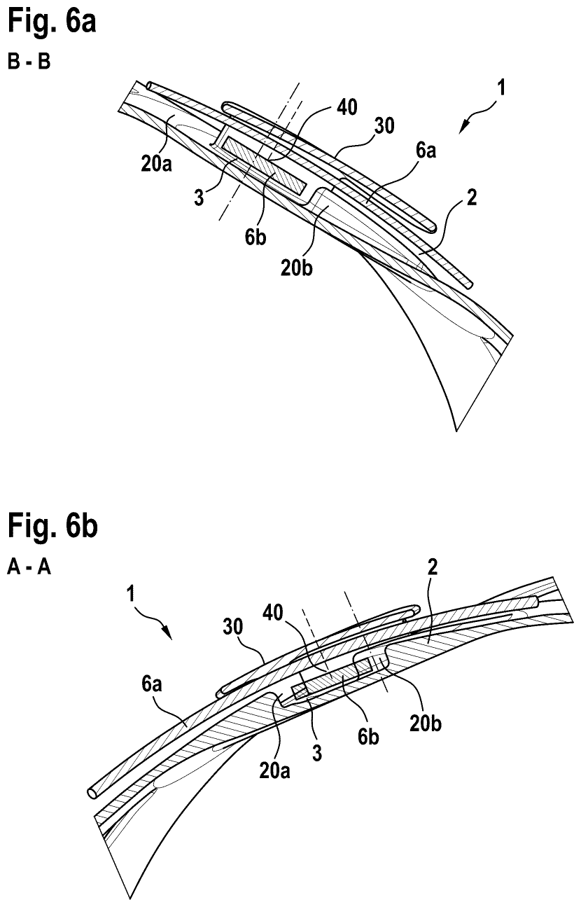

A section through the crossing element (1) shown in FIGS. 4a, 4b and 5 along the section line B-B is illustrated in FIG. 6a. FIG. 6b shows a section through the crossing element (1) shown in FIGS. 4a, 4b and 5 along the section line A-A.

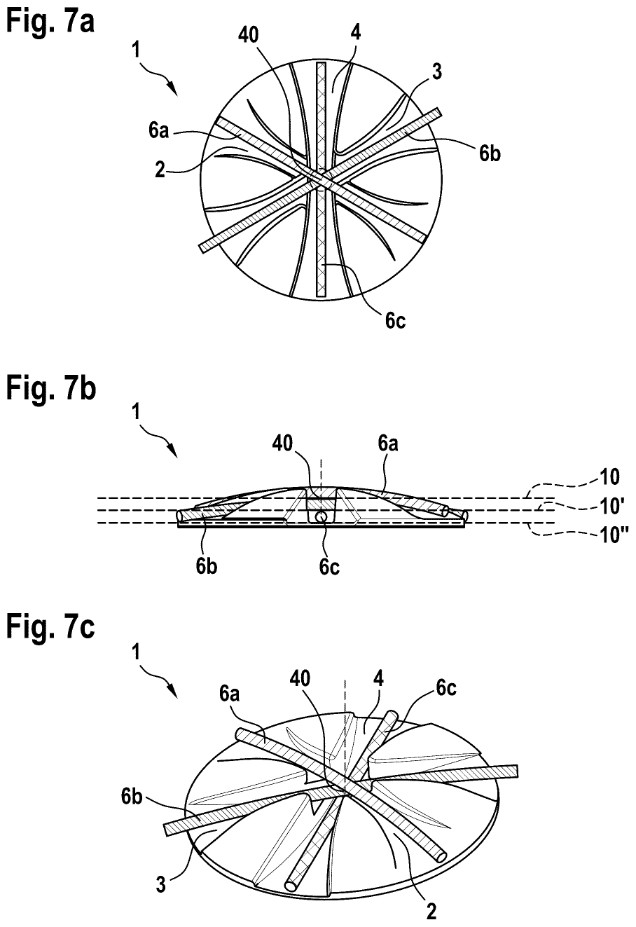

FIG. 7a shows a top view of a preferred embodiment of the crossing element (1) according to the invention with three guide sections (2, 3, 4) and tensioning elements (6a, 6b, 6c) guided therein, which cross at a central point (40). FIG. 7b shows a side view of the crossing element (1). The three tensioning elements (6a, 6b, 6c) are guided in the crossing point (40) in three different planes (10, 10′, 10″) so that the tensioning elements (6a, 6b, 6c) cross without contact. FIG. 7c shows a perspective view of the crossing element (1).

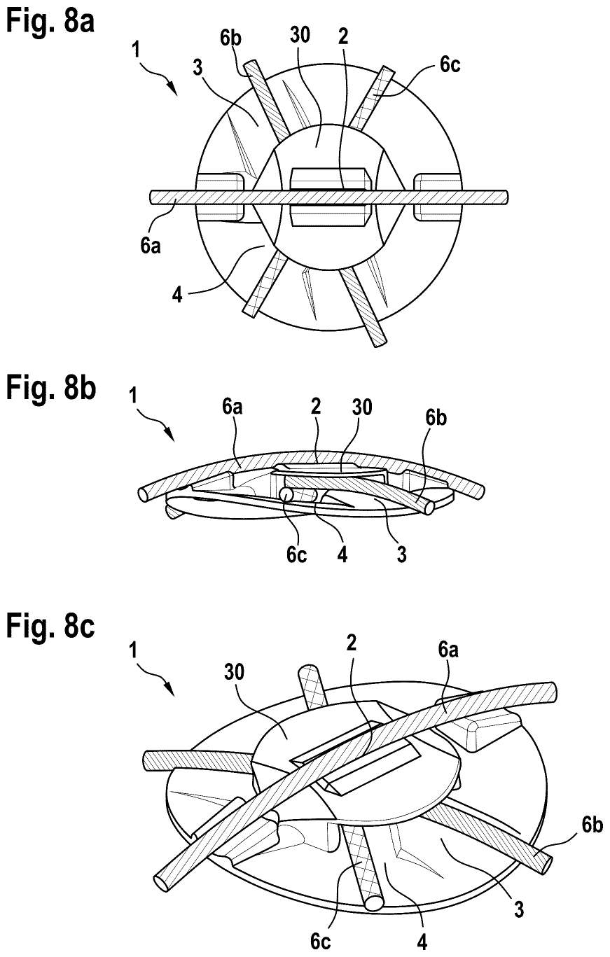

FIG. 8a shows a top view of a further embodiment of the crossing element (1) according to the invention. The crossing element (1) comprises a cover (30) in which a guide section (2) for receiving a tensioning element (6a) runs. The guide section (2) of the cover (30) guides the tensioning element (6a) above two tensioning elements (6b, 6c) running in the guide sections (3) and (4). The tensioning elements (6a, 6b, 6c) running in the guide sections (2, 3, 4) cross each other without contact. FIG. 8b shows a side view of the further embodiment of the crossing element (1). FIG. 8c shows a perspective view of the embodiment of the crossing element (1) shown in FIGS. 8a and 8b.

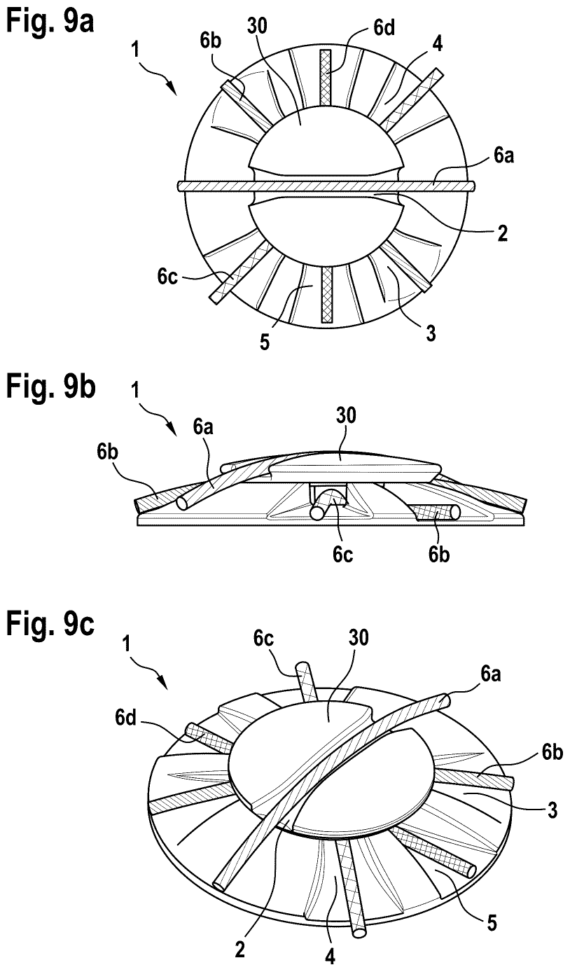

The embodiment of the crossing element (1) according to the invention shown in FIGS. 9a, 9b and 9c allows four tensioning elements (6 a, 6b, 6c, 6d) guided in the guide sections (2), (3), (4) and (5) to cross without contact. The guide section (2) runs in the cover (30) of the crossing element (1) in the form of a groove and guides the tensioning element (6a) above the tensioning elements (6b, 6c, 6d) running in three different planes below.

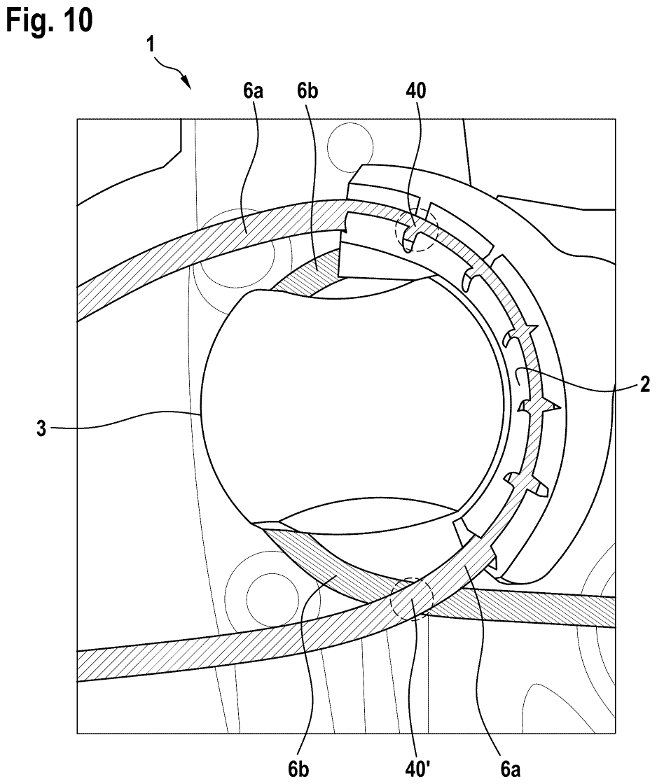

FIG. 10 shows a top view of a further embodiment of the crossing element (1) according to the invention. The embodiment shown is both a locking element and a crossing element (1) according to the invention, which comprises guide sections (2) and (3) that are arcuate in shape and run in different planes, in which tensioning elements (6a, 6b) are guided in such a way that they cross without contact at two different crossing points (40, 40′) of the crossing element (1). The tensioning element (6a) guided in the first guide section (2) runs in the two crossing points (40, 40′) above the tensioning element (6b) guided in the second guide section (3).

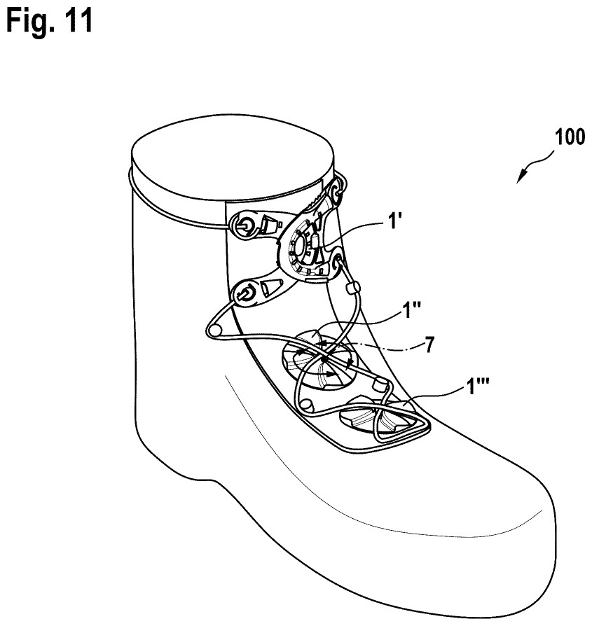

FIG. 11 shows a sports aid device (100) in the form of a shoe with three crossing elements (1′, 1″, 1″) in which tensioning elements cross without contact. In the embodiment shown, the second crossing element (1″) can rotate about a central axis of rotation (7).

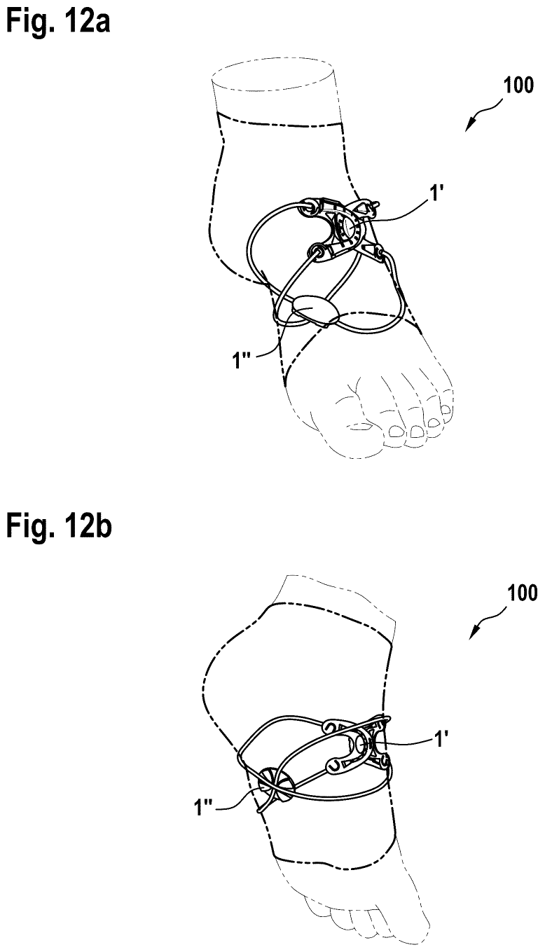

FIGS. 12a and 12b show an ankle joint orthosis/bandage (100) with two crossing elements (1′, 1″) from two different perspectives. When the ankle joint orthosis/bandage (100) is in place, the first crossing element (1′) is located on the back of the foot (instep) in the region of the metatarsal bones. The second crossing element (1″) is located opposite in the region of the medial arch or medial longitudinal arch of the sole of the foot.

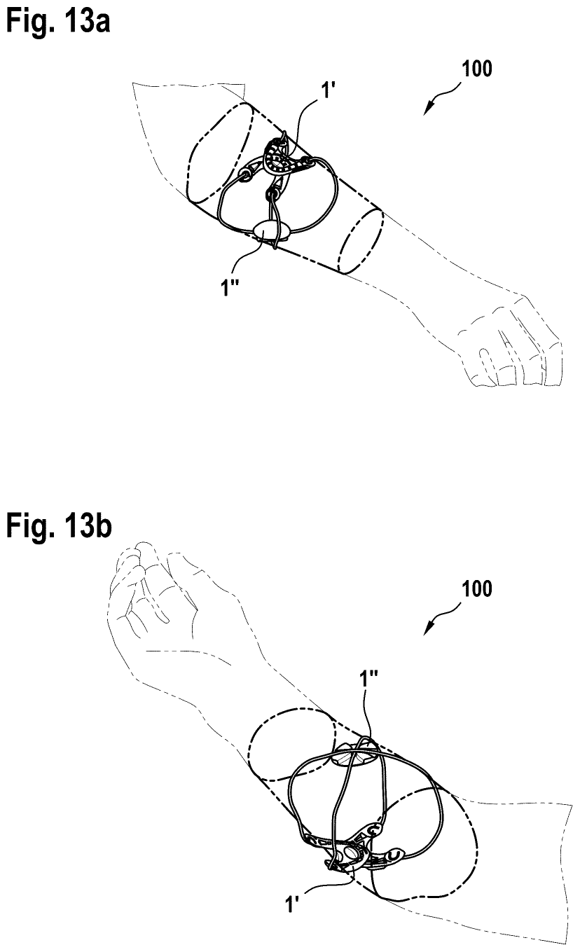

FIGS. 13a and 13b show a perspective view of a forearm orthosis/bandage (100) with two crossing elements (1′, 1″). When the forearm orthosis/bandage (100) is in place, the first crossing element (1′) is located on the outside of the forearm. The second crossing element (1″) is arranged opposite on the inside of the forearm.

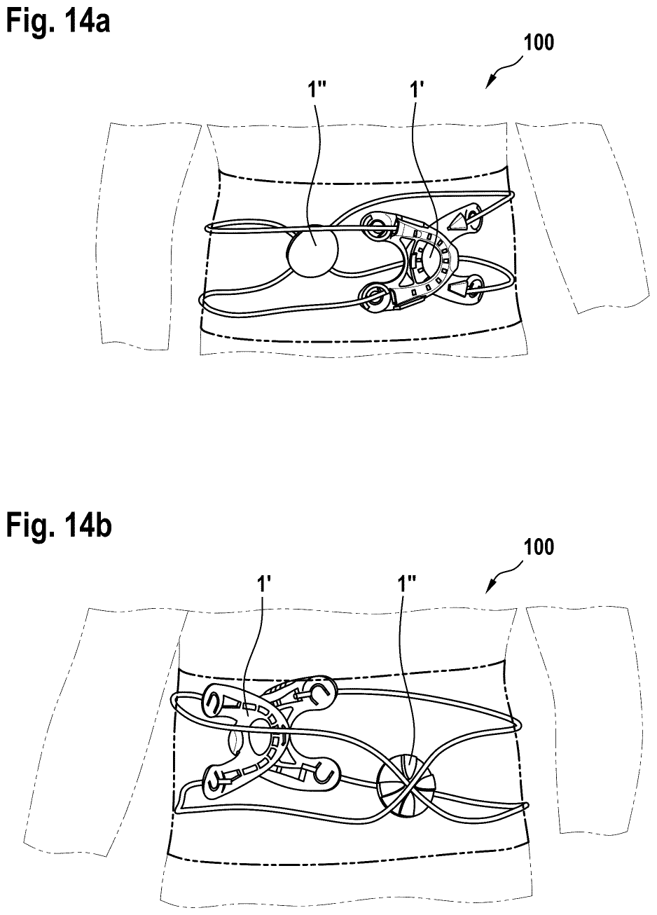

FIGS. 14a and 14b show different perspectives of a back orthosis/bandage (100) according to the invention with two crossing elements (1′, 1″) facing each other, in which tensioning elements cross without contact. In the embodiment shown, the first crossing element (1′) is located approximately at the level of the navel and the second crossing element (1″) is located on the side of the back in the region of the lumbar spine.

### LIST OF REFERENCE SIGNS

1 crossing element
1′ first crossing element
1″ second crossing element
1′″ third crossing element
2 first guide section
3 second guide section
4 third guide section
5 fourth guide section
6a tensioning element
6b tensioning element
6c tensioning element
6d tensioning element
7 axis of rotation
10 first plane
10′ second plane
10″ third plane
20a ramp
20b ramp
30 cover
40 crossing point
40′ crossing point
100 orthopedic or sports aid device

1. A crossing element for an orthopedic or sports aid device, comprising at least one first guide section for receiving a tensioning element of the aid device and at least one second guide section for receiving a tensioning element of the aid device, wherein the at least first and second guide sections of the crossing element are designed and arranged relative to one another such that a tensioning element guided in the first guide section and a tensioning element guided in the second guide section cross each other without contact at at least crossing one point.

2. The crossing The crossing element according to claim 1, wherein the first and/or the second guide section of the crossing element is a guide channel.

3. The crossing element according to claim 2, wherein the guide channel is designed in the form of a groove.

4. The crossing element according to claim 1, wherein the first and/or second guide section is designed such that a tensioning element guided in the first and/or second guide section is at least partially encompassed.

5. The crossing element according to claim 1, wherein the first and second guide sections are located in different planes at least in the a region of the at least one crossing point of the tensioning element guided in the first guide section with the tensioning element guided in the second guide section.

6. The crossing element according to claim 1, wherein the first guide section comprises two ramps, wherein the second guide section runs between the two ramps of the first guide section so that a tensioning element running in the first guide section is guided by means of the two ramps above a tensioning element running in the second guide section.

7. The crossing element according to claim 1, wherein the crossing element (comprises at least one cover above the at least one crossing point of the tensioning element guided in the first guide section with the tensioning element guided in the second guide section.

8. The crossing element according to claim 7, wherein the at least one cover of the crossing element comprises at least one further guide section in which a tensioning element of the aid device can be guided.

9. The crossing element according to claim 7, wherein the at least one cover is removable.

10. The crossing element according to claim 7, wherein the at least one cover is rotatably mounted.

11. The crossing element according to claim 1, wherein the tensioning element guided in the first guide section and the tensioning element guided in the second guide section are the same tensioning element of the aid device, in particular a single connected tensioning element of the aid device.

12. The crossing element according to claim 1, wherein the tensioning element guided in the first guide section and the tensioning element guided in the second guide section are different tensioning elements of the aid device, in particular separate tensioning elements of the aid device which are not connected to one another.

13. An orthopedic or sports aid device comprising at least one crossing element according to claim 1.

14. An orthopedic or sports aid device according to claim 13, wherein the aid device comprises at least one tensioning element, wherein the tensioning element is selected from the group consisting of tensioning or pulling strap, tensioning or pulling rope, tensioning or pulling band, and limb-like tensioning or pulling element.

15. The orthopedic or sports aid device according to claim 13, selected from the group consisting of back orthosis, back bandage, hip orthosis, hip bandage, knee joint orthosis, knee joint bandage, elbow orthosis, elbow bandage, arm orthosis, arm bandage, ankle joint orthosis, ankle joint bandage, gloves, and shoes.

16. The orthopedic or sports aid device according to claim 13, wherein the at least one crossing element is arranged rotatably mounted on the aid device.

17. The orthopedic or sports aid device according to claim 13, wherein the first and second guide sections of the at least one crossing element are arranged such that the tensioning elements guided therein cross at an angle of intersection.

18. The orthopedic or sports aid device according to claim 13, wherein the at least one crossing element comprises at least two different materials.

19. The orthopedic or sports aid device according to claim 13, wherein at least one of the guide sections of the at least one crossing element is designed as a groove tapering toward the at least one crossing point to restrict a freedom of movement of the tensioning element and prevent slippage at the crossing point.

20. An orthosis system, comprising:
an orthotic aid device for supporting a musculoskeletal system of a user of the orthotic aid device; and
a crossing element for receiving a first tensioning element of the orthotic aid device and a second tensioning element of the orthotic aid device, the crossing element comprising:
a first guide section for receiving the first tensioning element,
a second guide section for receiving the second tensioning element, the second guide section being arranged in a different plane relative to the first guide section such that the first tensioning element and the second tensioning element cross each other without contact.
